(19) United States
(12) Patent Application Publication
(10) Pub. No.: US 2008/0004619 A1
(43) Pub. Date: Jan. 3, 2008

(54) ELECTROSURGICAL BIPOLAR INSTRUMENT

(75) Inventors: Jerry L. Malis, King of Prussia, PA (US); Robert R. Acorcey, Cherry Hill, NJ (US); Anthony John Groch, Mantua, NJ (US)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: SYNERGETICS USA, INC., O'Fallon, MO (US)

(21) Appl. No.: 11/770,356

(22) Filed: Jun. 28, 2007

Related U.S. Application Data
(60) Provisional application No. 60/806,010, filed on Jun. 28, 2006.

(51) Int. Cl. A61B 18/14

(52) U.S. Cl. 606/42; 606/45; 606/48; 606/49; 606/50

(57) ABSTRACT

An electrosurgical bipolar instrument includes an instrument housing having a distal end, a proximal end and an elongated body therebetween. The electrosurgical bipolar instrument includes a pair of radio frequency (RF) electrodes disposed at the distal end of the instrument housing and a control circuit mounted within the instrument housing. The control circuit has a first cut/coagulate mode pad, a common pad and a first cut/coagulate mode push button configured to electrically couple the first cut/coagulate mode pad to the common pad when actuated. The electrosurgical bipolar instrument further includes a plug coupled to the proximal end of the instrument housing having first and second RF contacts electrically coupled to the pair of RF electrodes. The plug has a first cut/coagulate mode contact and a first common contact electrically isolated from the first and second RF contacts and the pair of RF electrodes.

### CROSS REFERENCE TO RELATED APPLICATIONS

This application claims the benefit of U.S. Provisional Patent Application No. 60/806,010 entitled “Electrosurgical Bipolar Instrument”, filed on Jun. 28, 2006.

### BACKGROUND OF THE INVENTION

Embodiments of the present invention relate to an electrosurgical bipolar instrument and, more particularly, an electrosurgical bipolar instrument having an isolation circuit.

Devices for controlling monopolar and bipolar electrodes are generally known in the art. For example, U.S. Pat. Nos. 4,590,934; 5,318,563; and 7,041,096 B2, the contents of which are all incorporated by reference herein, generally relate to bipolar radio frequency (RF) generators. The electrodes in the prior art systems are generally used for cutting, coagulating and stimulating tissue, and, in some instances, for taking measurements during any of the aforementioned procedures. Often, the electrodes are installed in an insulated instrument handle for safe handling by a user during use.

The output of the RF generator is an electrical waveform controlled by RF modulation. The waveforms of such prior art generators and bipolar electrosurgical instruments provide adequate cutting, coagulating and stimulating without undesirable secondary or collateral damage.

It is desirable to provide an electrosurgical bipolar instrument having an isolation control circuit. It is also desirable to provide an electrosurgical bipolar instrument having control circuitry which is isolated from RF electrodes, so that the electrosurgical bipolar instrument is not susceptible to noise or induced to generate leakage current.

### BRIEF SUMMARY OF THE INVENTION

Briefly stated, an embodiment of the present invention comprises an electrosurgical bipolar instrument. The electrosurgical bipolar instrument includes an instrument housing having a distal end, a proximal end and an elongated body therebetween. The electrosurgical instrument also comprises a control circuit mounted within the instrument housing, the control circuit having a first cut/coagulate mode pad, a common pad and a first cut/coagulate mode pushbutton configured to electrically couple the first cut/coagulate mode pad to the common pad when actuated. The electrosurgical bipolar instrument also comprises a plug coupled to the proximal end of the instrument housing, the plug having first and second radiofrequency (RF) contacts and a pair of radiofrequency (RF) electrodes disposed at the distal end of the instrument housing. The pair of RF electrodes are electrically coupled with the first and second RF contacts and electrically isolated from the control circuit.

Another embodiment of the electrosurgical bipolar instrument comprises an instrument housing having a distal end, a proximal end and an elongated body therebetween, a control circuit mounted within the instrument housing and comprising means for initiating a control signal, and associated wiring and a cable for electrically coupling the control circuit to a radiofrequency (RF) generator. The instrument also comprises a pair of RF electrodes disposed proximate the distal end of the instrument housing and RF conductors and a cable for electrically coupling the RF electrodes to the RF generator. Further the instrument also includes associated insulation/separation for electrically isolating the control circuit and the associated wiring for electrically coupling the control circuit to the RF generator from the RF electrodes and the RF conductors for coupling the RF electrodes to the RF generator.

Another embodiment of the present invention includes a method of operating a bipolar instrument. The method comprises electrically coupling the bipolar instrument to a radiofrequency (RF) generator. The bipolar instrument includes a housing, a control circuit within the housing, and a pair of RF electrodes coupled to the housing. The control circuit and the RF electrodes are electrically coupled to the RF generator and are electrically isolated from one another external to the RF generator. The method also comprises initiating a control signal from the control circuit by user actuation to control an output from the RF generator, thereby effecting control of electrical characteristics between the pair of RF electrodes.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a perspective view of an electrosurgical bipolar instrument in accordance with a preferred embodiment of the present invention;

FIG. 2 is a right side elevational view of the electrosurgical bipolar instrument of FIG. 1;

FIG. 3 is a left side elevational view of the electrosurgical bipolar instrument of FIG. 1;

FIG. 4 is a bottom plan view of the electrosurgical bipolar instrument of FIG. 1;

FIG. 5 is a top plan view of the electrosurgical bipolar instrument of FIG. 1;

FIG. 6 is a rear elevational view of the electrosurgical bipolar instrument of FIG. 1;

FIG. 7A is an exploded perspective view of the electro surgical bipolar instrument of FIG. 1;

FIG. 7B is a cross-sectional elevational view of a portion of the electrosurgical bipolar instrument of FIG. 1;

FIG. 8 is a top plan view of a control circuit for the electrosurgical bipolar instrument of FIG. 1; and

FIGS. 9-10 are exemplary control circuits for an RF generator for use with preferred embodiments of the present invention.

### DETAILED DESCRIPTION OF THE INVENTION

Certain terminology is used in the following description for convenience only and is not limiting. The words “right,” “left,” “lower,” and “upper” designate directions in the drawings to which reference is made. The words “inwardly” and “outwardly” refer to direction toward and away from, respectively, the geometric center of the object described and designated parts thereof. The terminology includes the words above specifically mentioned, derivatives thereof and words of similar import. Additionally, the words “a” and “an,” as used in the claims in the corresponding portions of the specification, mean “at least one.”

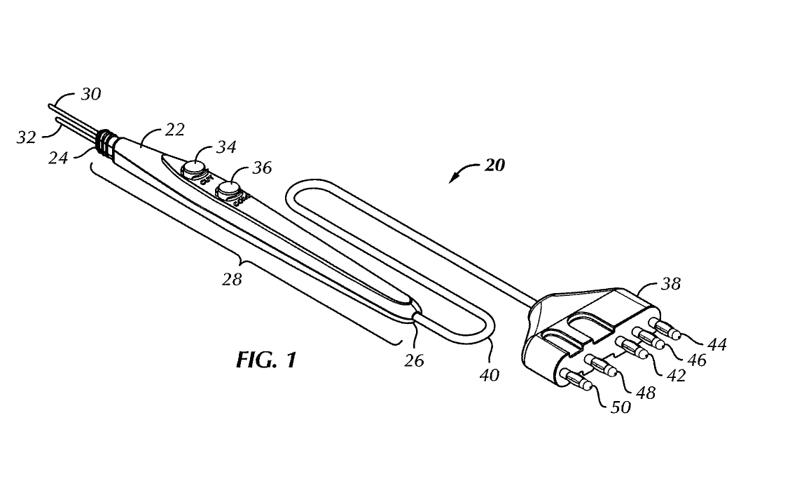

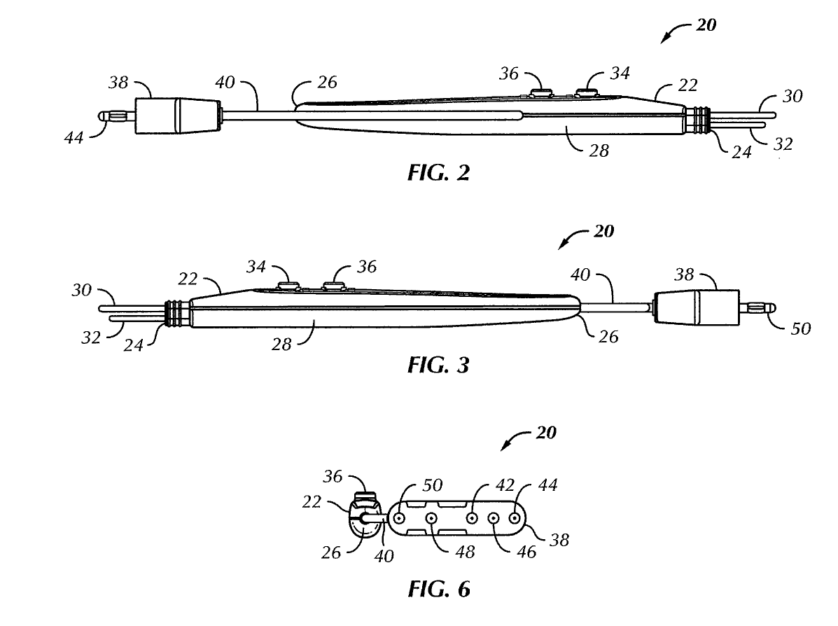

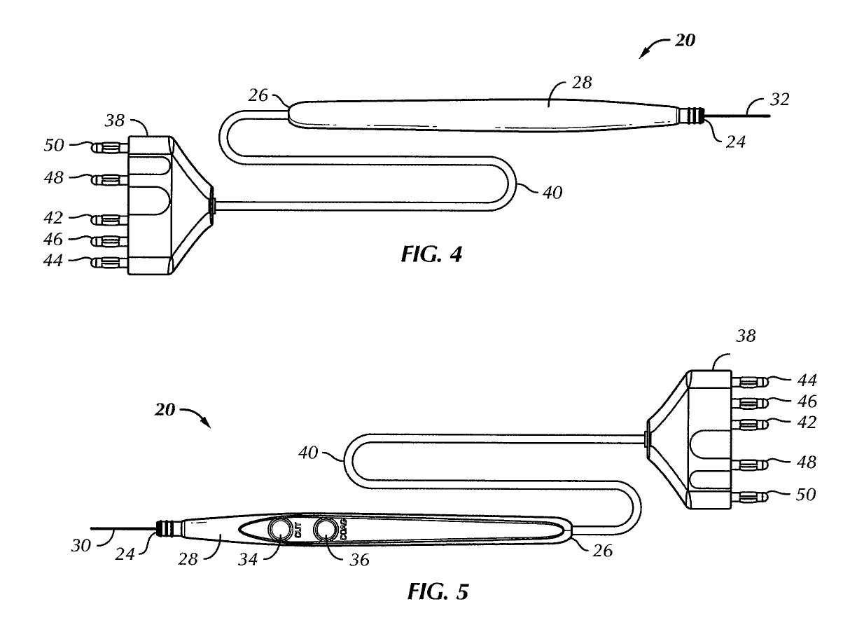

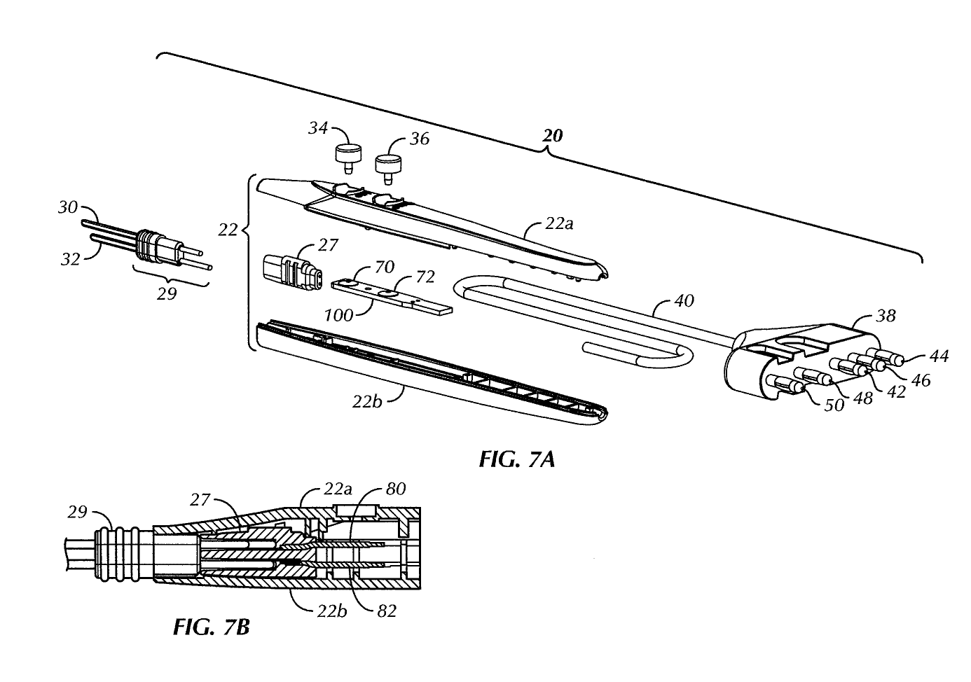

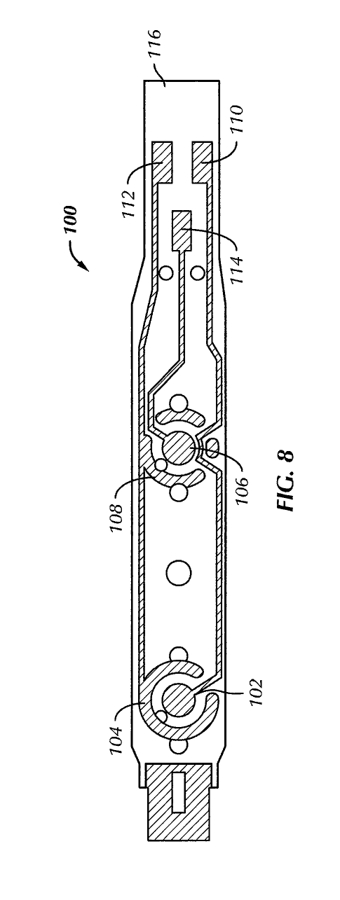

Referring to the drawings in detail, wherein like numerals indicate like elements throughout, there is shown in FIGS. 1-6 and 7A and 7B an electrosurgical bipolar instrument 20 in accordance with a preferred embodiment of the present invention. The electrosurgical bipolar instrument 20 includes an instrument housing 22 having a distal end 24, a proximal end 26 and an elongated body 28 therebetween. First and second cut/coagulate mode push buttons 34 and 36 are located on the upper surface of the instrument housing 22. The electrosurgical bipolar instrument 20 includes a pair of radio frequency (RF) electrodes 30, 32 disposed in an electrode housing 29 that is disposed at the distal end 24 of the instrument housing 22. The electrode housing 29 is detachably connected to a connection housing 27 that is disposed in the instrument housing 22 proximate the distal end 24 of the instrument housing 22. Different electrode housings 29 with different electrodes 30, 32 can be interchangeably connected to the connection housing 27. A control circuit 100 (FIG. 8) is mounted within the instrument housing 22, as best shown in the exploded view of FIG. 7A. The electrosurgical bipolar instrument 20 further includes a plug 38 coupled to the proximal end 26 of the instrument housing 22 by a cable 40. The plug 38 has first and second RF contacts 48, 50 electrically coupled to the pair of RF electrodes 30, 32 via the cable 40. The plug 38 also includes a first cut/coagulate mode contact 42, a second cut/coagulate mode contact 46 and a common contact 44. The housing 22 has an upper housing member 22a and a lower housing member 22b (FIG. 7A) which encases the control circuit 100 and associated wiring (not shown) that connects the plug 38 to the RF electrodes 30, 32. High voltage electrical insulation such as insulating tape (not shown), such as a tape made from DuPont Kapton®, or an equivalent, is used to insulate the associated wiring (not shown) that connects the plug 38 with the electrodes 30, 32 where the associated wiring (not shown) is disposed in the lower housing member 22b. Further, plug 38 can also include an additional contact (not shown) to connect the electrosurgical bipolar instrument 20 to an instrument ground or safety ground.

Referring to FIG. 7B, the electrode housing 29 connects detachably with the connection housing 27 (seen in cross-section) which is disposed proximate the distal end 24 of instrument housing 22 (seen in cross-section), between the upper housing 22a and the lower housing 22b. A pair of RF conductors 80, 82 (partially shown) are connected to the connection housing 27. Connection housing 27 is configured so that RF conductors 80, 82 are in electrical connection with the electrode housing 29 and the RF electrodes 30, 32 when the electrode housing 29 is connected to the connection housing 27. RF conductors 80, 82 are in electrical connection with the RF contacts 48, 50 of plug 38. RF conductors 80, 82 do not make physical contact with the control circuit 100.

Referring to FIG. 8, the control circuit 100 includes a first cut/coagulate mode pad 102, a first common pad 104, a second cut/coagulate mode pad 106 and a second common pad 108. The first cut/coagulate mode pad 102 is coupled to the first cut/coagulate mode plug contact 42 of plug 38 via conductive tracing 110 on a printed circuit board (PCB) 116. The first and second common pads 104, 108 are coupled to the common plug contact 44 of plug 38 via conductive tracing 112 on the PCB 116. The second cut/coagulate mode pad 106 is coupled to the second cut/coagulate mode plug contact 46 of plug 38 via conductive tracing 114 on the PCB 116. As shown in FIG. 7A, the control circuit 100 also includes a resilient flexible contact 70 disposed above the first cut/coagulate mode pad 102 and the first common mode pad 104. Control circuit 100 also includes a resilient flexible contact 72 that is disposed above the second cut/coagulate mode pad 106 and the second common mode pad 108. The cable 40 includes a plurality of conductors (not shown in detail) which are individually isolated to connect the conductive tracings 110, 112, 114 to the mode plug contacts 42, 44, 46.

The first cut/coagulate mode pad 102 and the first contact 42 are electrically isolated from the first and second RF contacts 48, 50 and the pair of RF electrodes 30, 32. Likewise, the second cut/coagulate mode pad 106 and the second cut/coagulate mode contact 46 are electrically isolated from the first and second RF contacts 48, 50 and the pair of RF electrodes 30, 32. Preferably, the PCB 116 is a multi-layer board having appropriate insulation and grounding layers.

The first cut/coagulate mode push button 34 in combination with resilient flexible contact 70 is configured to electrically couple or contact the first cut/coagulate mode pad 102 to the first common pad 104 when actuated or depressed by a user. Similarly, the second cut/coagulate mode push button 36 in combination with resilient flexible contact 72 is configured to electrically couple the second cut/coagulate mode pad 106 to the second common pad 108 when actuated or depressed by a user. A user of the electrosurgical instrument 20 initiates an isolated control signal, described in further detail below, to the RF generator controller (not shown) that corresponds to the first or second cut/coagulate mode by operating push buttons 34 and 36, respectively. In a different embodiment, push buttons 34, 36 could be used in combination with a conducting surface and a spring return, or the like, and thus electrically couple the mode pads 102, 104 or 106, 108 without the need of the resilient flexible contacts 70, 72.

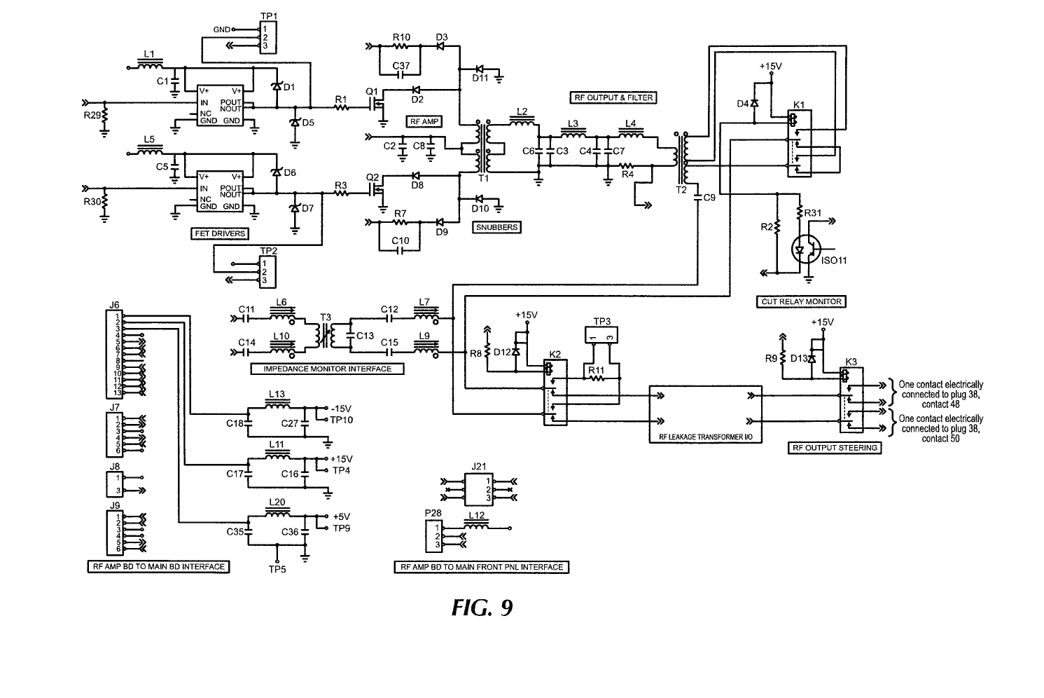

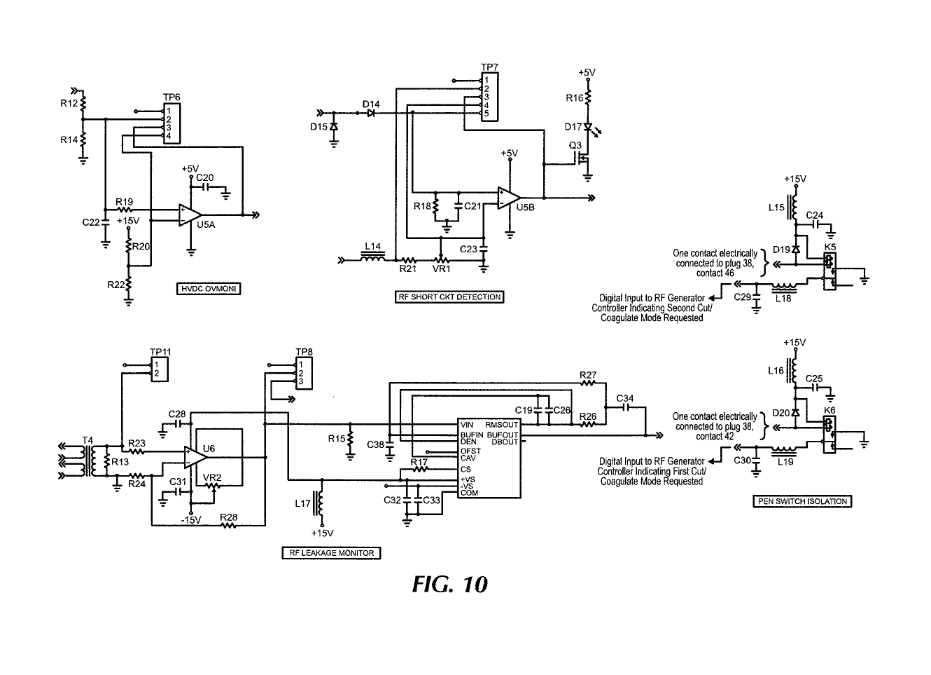

The plug 38 is adapted to plug into an RF generator having circuitry such as that depicted in the schematics shown in FIGS. 9-10. The first and second RF contacts 48, 50 are coupled directly to an RF output without interference or interruption in the instrument 20. The conductors 80, 82 (FIG. 7B) carrying the RF output from the plug 38 are connected to the connection housing 27. The connection housing 27 is configured to place the RF output conductors 80, 82 into electrical connection with the electrode housing 29. The electrodes 30, 32 are placed into electrical connection with the RF output when the electrode housing 29 is connected to the connection housing 27.

The first and second cut/coagulate mode contacts 42 and 46 and the common contact 44 provide inputs to a control circuit within the RF generator that controls the RF output to the RF contacts 48, 50. For example, the common 44 can apply voltage or ground (power supply ground or direct current ground) and by depressing the first or second cut/coagulate mode push buttons 34, 36 the common contact 44 is applied to either the first cut/coagulate mode contact 42 or to the second cut/coagulate mode contact 46 to actuate a relay, electronic switching device or the like in order to switch to/from a cut mode to a coagulate mode or vice versa all within the control circuitry shown on FIGS. 9-10. For the particular embodiment disclosed in FIG. 10, the common contact 44 applies direct current ground.

The low level inputs do not interrupt the RF signal applied to the electrodes 30, 32. Leakage current and/or noise in the electrosurgical instrument 20 can be prevented by not interfering with the RF signal on the electrodes 30, 32 by partial switching, bad contacts or the like. The control circuitry changes the mode of operation within the driving circuitry RF output and filter directly based upon the inputs from the first and second cut/coagulate mode contacts 42, 46. As seen in FIG. 10, when the first cut/coagulate mode contact 42 is activated through the control circuit 100 as described above, a first isolation relay K6 is activated thereby providing a first isolated digital input to the controller of the RF generator (not shown) indicating the operator of the electrosurgical bipolar instrument 20 requires a RF output consistent with the first (preselected) cut/coagulate mode. The RF generator controller then provides a RF output consistent with the first cut/coagulate mode to the contacts 48, 50 of plug 38. Further, when the second cut/coagulate mode contact 46 is activated through the control circuit 100 as described above, a second isolation relay K5 is activated thereby providing a second isolated digital input to the controller of the RF generator (not shown) indicating the operator of the electrosurgical bipolar instrument 20 requires a RF output consistent with the second (preselected) cut/coagulate mode. The RF generator controller then provides a RF output consistent with the second cut/coagulate mode to the contacts 48, 50 of plug 38.

Another preferred embodiment of the present invention provides a method of operating the electrosurgical bipolar instrument 20. The method includes electrically coupling the electrosurgical bipolar instrument 20 to a RF generator (not shown). The electrosurgical bipolar instrument 20 includes a housing 22, a control circuit 100 disposed within the housing 22 and a pair of RF electrodes 30, 32 coupled to the housing 22. The control circuit 100 and the RF electrodes 30, 32 are electrically coupled to the RF generator and the control circuit 100 and the RF electrodes 30, 32 are also electrically isolated from one another external to the RF generator. The method further includes initiating a control signal from the control circuit 100 by user actuation to control an output from the RF generator. The user initiated control signal thereby effects control of the electrical characteristics between the pair of RF electrodes 30, 32. In a further embodiment of the method of operating the electrosurgical bipolar instrument 20, the initiating of the control signal is accomplished by actuating the first cut/coagulate mode pushbutton 34 which is configured to electrically couple with the control circuit 100.

Another embodiment of the method described above includes placing the RF electrodes 30, 32 into contact with tissue (not shown) to perform a surgical procedure, for example a dental or a neurological procedure, or to perform some other medical procedure. The steps of the disclosed method embodiments are not necessarily carried out in the order disclosed. Further, the method steps are not limited to the steps disclosed as modifications of the disclosed method embodiments that are within the spirit and scope of the disclosed embodiments are also included.

From the foregoing, it can be seen that embodiments of the present invention are directed to an electrosurgical bipolar instrument having an isolation control circuit and methods for using the electrosurgical bipolar instrument. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the embodiments of the present invention as defined by the appended claims.

What is claimed is:

1. An electrosurgical bipolar instrument comprising:
an instrument housing having a distal end, a proximal end and an elongated body therebetween;
a control circuit mounted within the instrument housing, the control circuit having a first cut/coagulate mode pad, a common pad and a first cut/coagulate mode pushbutton configured to electrically couple the first cut/coagulate mode pad to the common pad when actuated;
a plug coupled to the proximal end of the instrument housing, the plug having first and second radiofrequency (RF) contacts; and
a pair of RF electrodes disposed proximate the distal end of the instrument housing, the pair of RF electrodes electrically coupled with the first and second RF contacts and electrically isolated from the control circuit.

2. The electrosurgical bipolar instrument of claim 1, wherein the plug has a first cut/coagulate mode contact electrically coupled to the first cut/coagulate mode pad of the control circuit and has a common contact electrically coupled to the common pad of the control circuit.

3. The electrosurgical bipolar instrument of claim 2, further comprising a pair of RF conductors electrically connecting the first and second RF contacts with the pair of RF electrodes, the control circuit being electrically isolated from the pair of RF conductors.

4. The electrosurgical bipolar instrument of claim 3, wherein the pair of RF conductors and the control circuit are physically separated by high voltage insulation disposed between the control circuit and the pair of RF conductors.

5. The electrosurgical bipolar instrument of claim 2, wherein the control circuit comprises a multilayered printed circuit board (PCB).

6. The electrosurgical bipolar instrument of claim 5, wherein at least one layer of the PCB is composed of an electrically insulating material.

7. The electrosurgical bipolar instrument of claim 2, wherein the control circuit further comprises a second cut/coagulate mode pad and a second cut/coagulate mode pushbutton configured to electrically couple the second cut/coagulate mode pad to the common pad when actuated, and wherein the plug has a second cut/coagulate mode contact electrically coupled to the second cut/coagulate mode pad of the control circuit, the second cut/coagulate mode contact being electrically isolated from the first and second RF contacts and the pair of RF electrodes.

8. The electrosurgical bipolar instrument of claim 2, wherein the RF electrodes are disposed in an electrode housing, the electrode housing disposed at the distal end of the instrument housing and detachably connected to a connection housing, the connection housing disposed proximate the distal end of the instrument housing.

9. An electrosurgical bipolar instrument comprising:
an instrument housing having a distal end, a proximal end and an elongated body therebetween;
a control circuit mounted within the instrument housing and comprising means for initiating a control signal;
means for electrically coupling the control circuit to a radiofrequency (RF) generator;
a pair of RF electrodes disposed proximate the distal end of the instrument housing;
means for electrically coupling the RF electrodes to the RF generator; and
means for electrically isolating the control circuit and the means for electrically coupling the control circuit to the RF generator from the RF electrodes and the means for coupling the RF electrodes to the RF generator.

10. The electrosurgical bipolar instrument of claim 9, wherein the control circuit comprises a multilayered printed circuit board (PCB).

11. The electrosurgical bipolar instrument of claim 10, wherein at least one layer of the PCB is composed of an electrically insulating material.

12. The electrosurgical bipolar instrument of claim 9, wherein the means for initiating a control signal comprises a first cut/coagulate mode pushbutton configured to electrically couple a first cut/coagulate mode pad to a common pad when actuated.

13. The electrosurgical bipolar instrument of claim 12, wherein the means for initiating a control signal further comprises a second cut/coagulate mode pushbutton configured to electrically couple a second cut/coagulate mode pad to the common pad when actuated.

14. The electrosurgical bipolar instrument of claim 9, wherein the means for electrically isolating comprises physical separation by high voltage insulation disposed between the control circuit and the pair of RF conductors.

15. A method of operating a bipolar instrument comprising:
electrically coupling the bipolar instrument to a radiofrequency (RF) generator, the bipolar instrument including a housing, a control circuit within the housing, and a pair of RF electrodes coupled to the housing, the control circuit and the RF electrodes being electrically coupled to the RF generator and being electrically isolated from one another external to the RF generator; and
initiating a control signal from the control circuit by user actuation to control an output from the RF generator, thereby effecting control of electrical characteristics between the pair of RF electrodes.

16. The method of claim 15, wherein initiating a control signal comprises actuating a first cut/coagulate mode pushbutton configured to electrically couple with the control circuit.

17. The method of claim 15, further comprising contacting tissue with the pair of RF electrodes to perform a surgical procedure.
